SEQUENCE LISTING (4 sequences)

SEQ ID NO: 1 - DNA, 28 bp, Unknown
  primer/probe
ctccatcagttgctggagggtgtcatta

SEQ ID NO: 2 - DNA, 29 bp, Unknown
  primer
gaaggtttatcttttgtccttgctgctca

SEQ ID NO: 3 - DNA, 24 bp, Unknown
  primer
ataatcacatggagagccacaagc

SEQ ID NO: 4 - DNA, 24 bp, Unknown
  primer
gcacttctttggtatctgagaaag

(19) United States
(12) Patent Application Publication
(10) Pub. No.: US 2005/0193431 A1
(43) Pub. Date: Sep. 1, 2005

(54) Somatic cell line

(76) Inventors: Yann Echelard, Jamaica Plains, MA (US); Esmail Behboodi, Shrewsbury, MA (US); David Melican, Fiskdale, MA (US); Carol Ziomek, Milford, MA (US); William Gavin, Dudley, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/660,384

(22) Filed: Sep. 11, 2003

Related U.S. Application Data
(62) Division of application No. 09/298,508, filed on Apr. 22, 1999, now abandoned.
(60) Provisional application No. 60/106,728, filed on Nov. 2, 1998.

(51) Int. Cl. A01K 67/027; C12N 5/06

(52) U.S. Cl. 800/14; 800/7; 435/325

(57) ABSTRACT

The invention features a purified embryonic or fetal caprine somatic cell. The somatic cell can be in a preparation of embryonic or fetal caprine somatic cells. In addition, the cell can be used to derive an embryonic or fetal caprine somatic cell line. The somatic cell can be a genetically engineered caprine somatic cell, e.g., a somatic cell which includes a transgenic sequence, or which a nucleic acid has been introduced.

### SUMMARY OF THE INVENTION

The present invention is based, in part, on the discovery that cloned and transgenic mammals, e.g., cloned and transgenic goats, can be produced by introduction of a somatic cell chromosomal genome into a functionally enucleated oocyte. The mammals can be embryonic, fetal or post natal, e.g., an adult mammal.

The use somatic cell lines, e.g., recombinant primary somatic cell lines, for nuclear transfer of transgenic nuclei dramatically increases the efficiency of production of transgenic animals, e.g., up to 100%, if the animals are made by certain methods. It also solves the initial mosaicism problem as each cell in the developing embryo contains the transgene. In addition, using nuclear transfer from transgenic cell lines to generate transgenic animals, e.g., transgenic goats, permits an accelerated scale up of a specific transgenic line. For example, a herd can be scaled up in one breeding season.

The generation of transgenic animals, e.g., transgenic goats, by nuclear transfer with somatic cells has the additional benefit of allowing genetic manipulations that are not feasible with traditional microinjection approaches. For example, nuclear transfer with somatic cells allows the introduction of specific mutations, or even the targeting of foreign genes directed to specific sites in the genome solving the problem of integration position effect. Homologous recombination in the donor somatic cells can “knock-out” or replace the endogenous protein, e.g., a endogenous goat protein, to lower purification costs of heterologous proteins expressed in milk and help to precisely adjust the animal bioreactors.

Accordingly, in one aspect, the invention features, an embryonic or fetal caprine somatic cell.

In a preferred embodiment, the cell is a purified embryonic or fetal caprine somatic cell.

In a preferred embodiment, the cell is in a preparation of embryonic or fetal caprine somatic cells.

In a preferred embodiment, the cell can be used to derive an embryonic or fetal caprine somatic cell line.

In a preferred embodiment, the cell includes a transgene, e.g., a transgene encoding a polypeptide. The transgene can be: integrated into the genome of the somatic cell; a heterologous transgene, e.g., a heterologous transgene which includes a human sequence; a knockout, knockin or other event which disrupts the expression of a caprine gene; a sequence which encodes a protein, e.g., a human protein; a heterologous promoter; a heterologous sequence under the control of a promoter, e.g., a caprine promoter. The transgenic sequence can encode a product of interest such as a protein, polypeptide or peptide.

In a preferred embodiment, the transgene encodes any of: a hormone, an immunoglobulin, a plasma protein, and an enzyme. The transgene can encode, e.g., any of: α-1 proteinase inhibitor, alkaline phosphotase, angiogenin, extracellular superoxide dismutase, fibrogen, glucocerebrosidase, glutamate decarboxylase, human serum albumin, myelin basic protein, proinsulin, soluble CD4, lactoferrin, lactoglobulin, lysozyme, lactoalbumin, erythrpoietin, tissue plasminogen activator, human growth factor, antithrombin III, insulin, prolactin, and α1-antitrypsin.

In a preferred embodiment, the transgene is under the control of a promoter, e.g., a heterologous or a caprine promoter. The promoter can be a tissue-specific promoter. The tissue specific promoter can be any of: a milk-specific promoter; a blood-specific promoter; a muscle-specific promoter; a neural-specific promoter; a skin-specific promoter; a hair specific promoter; and, a urine-specific promoter. The milk-specific promoter can be, e.g., any of: a β-casein promoter; a β-lactoglobin promoter; a whey acid protein promoter; and a lactalbumin promoter.

In a preferred embodiment, the somatic cell is a fibroblast. The fibroblast can be a primary fibroblast or a primary derived fibroblast.

In a preferred embodiment, the cell is obtained from a goat, e.g., an embryonic goat, derived from a germ cell obtained from a transgenic mammal. The germ cell can be sperm from a transgenic goat.

In a preferred embodiment, the cell is a genetically engineered embryonic or fetal caprine somatic cell, e.g., a purified genetically engineered embryonic or fetal caprine somatic cell.

In a preferred embodiment, the cell is part of a preparation of genetically engineered embryonic or fetal caprine somatic cells. In another preferred embodiment, the cell is used to derive a genetically engineered embryonic or fetal caprine somatic cell line.

In a preferred embodiment, the genetically engineered cell includes a nucleic acid, e.g., a nucleic acid encoding a polypeptide, which has been introduced into the cell. The nucleic acid can be: integrated into the genome of the somatic cell; a heterologous nucleic acid, e.g., a heterologous nucleic acid which includes a human sequence; a knockout, knockin or other event which disrupts the expression of a caprine gene; a sequence which encodes a protein, e.g., a human protein; a heterologous promoter; a heterologous sequence under the control of a promoter, e.g., a caprine promoter. The nucleic acid sequence can encode any product of interest such as a protein, polypeptide or peptide.

In a preferred embodiment, the nucleic acid encodes any of: a hormone, an immunoglobulin, a plasma protein, and an enzyme. The nucleic acid can encode, e.g., any of: α-1 proteinase inhibitor, alkaline phosphotase, angiogenin, extracellular superoxide dismutase, fibrogen, glucocerebrosidase, glutamate decarboxylase, human serum albumin, myelin basic protein, proinsulin, soluble CD4, lactoferrin, lactoglobulin, lysozyme, lactoalbumin, erythrpoietin, tissue plasminogen activator, human growth factor, antithrombin m, insulin, prolactin, and α1-antitrpsin.

In a preferred embodiment, the nucleic acid is under the control of a promoter, e.g., a caprine or heterologous promoter. The promoter can be a tissue-specific promoter. The tissue specific promoter can be any of: a milk-specific promoter; a blood-specific promoter; a muscle-specific promoter; a neural-specific promoter; a skin-specific promoter; a hair specific promoter; and, a urine-specific promoter. The milk-specific promoter can be, e.g., any of: a β-casein promoter; a β-lactoglobin promoter; a whey acid protein promoter; and a lactalbumin promoter.

In a preferred embodiment, the somatic cell is a fibroblast. The fibroblast can be a primary fibroblast or a primary derived fibroblast.

In a preferred embodiment, the cell is obtained from a goat, e.g., an embryonic goat, derived from a germ cell obtained from a transgenic goat. The germ cell can be sperm or an oocyte from a transgenic goat.

In a preferred embodiment, the cell is used as a source of genetic material for nuclear transfer.

In another aspect, the invention features an embryonic or fetal caprine somatic cell, a preparation of cells, or an embryonic or fetal caprine somatic cell line, e.g., as described herein, in a container, e.g., an airtight or liquid tight container.

In another aspect, the invention features an embryonic or fetal caprine somatic cell, a preparation of cells, or an embryonic or fetal caprine somatic cell line, e.g., as described herein, which is frozen, e.g., is cryopreserved.

In another aspect, the invention features a kit. The kit includes a container of the cell or cells described herein. In a preferred embodiment, the kit further includes instructions for use in preparing a transgenic animal.

In a preferred embodiment, the kit further includes a recipient oocyte, e.g., an enucleated oocyte.

In another aspect, the invention features a method for providing a component for the production of a cloned or transgenic goat. The method includes obtaining a frozen sample of the cell or cells, e.g., those described herein, and thawing the sample.

In another aspect, the invention features, a method of preparing an embryonic or fetal caprine somatic cell line. The method includes obtaining a somatic cell from an embryonic or fetal goat; and, culturing the cell, e.g., in a suitable medium, such that a somatic cell line is obtained.

In a preferred embodiment, the cell line is a genetically engineered cell line, e.g., the cell comprises a transgene. The transgene can be: integrated into the somatic cell genome; a heterologous transgene, e.g., a heterologous transgene which includes a human sequence; a knockout, knockin or other event which disrupts the expression of a caprine gene; a sequence which encodes a protein, e.g., a human protein; a heterologous promoter; a heterologous sequence under the control of a promoter, e.g., a caprine promoter. The transgenic sequence can encode any product of interest such as a protein, polypeptide or peptide.

In a preferred embodiment, the transgene encodes any of: a hormone, an immunoglobulin, a plasma protein, and an enzyme. The transgene can encode any protein, e.g., any of: α-1 proteinase inhibitor, alkaline phosphotase, angiogenin, extracellular superoxide dismutase, fibrogen, glucocerebrosidase, glutamate decarboxylase, human serum albumin, myelin basic protein, proinsulin, soluble CD4, lactoferrin, lactoglobulin, lysozyme, lactoalbumin, erythrpoietin, tissue plasminogen activator, human growth factor, antithrombin III, insulin, prolactin, and α1-antitrypsin.

In a preferred embodiment, the transgene is under the control of a promoter, e.g., a caprine or heterologous promoter. The promoter can be a tissue-specific promoter. The tissue specific promoter can be any of: a milk-specific promoter; a blood-specific promoter; a muscle-specific promoter; a neural-specific promoter; a skin-specific promoter; a hair specific promoter; and, a urine-specific promoter. The milk-specific promoter can be, e.g., any of: a β-casein promoter; a β-lactoglobin promoter; a whey acid protein promoter; and a lactalbumin promoter.

In a preferred embodiment, the genetically engineered cell includes a nucleic acid, e.g., a nucleic acid encoding a polypeptide, which has been introduced into the cell. The nucleic acid can be: integrated into the genome of the somatic cell; a heterologous nucleic acid, e.g., a heterologous nucleic acid which includes a human sequence; a knockout, knockin or other event which disrupts the expression of a-caprine gene; a sequence which encodes a protein, e.g., a human protein; a heterologous promoter; a heterologous sequence under the control of a promoter, e.g., a caprine promoter. The nucleic acid sequence can encode any product of interest such as a protein, polypeptide or peptide.

In a preferred embodiment, the nucleic acid can encode any of: a hormone, an immunoglobulin, a plasma protein, and an enzyme. The nucleic acid can encode, e.g.,. any of: α-1 proteinase inhibitor, alkaline phosphotase, angiogenin, extracellular superoxide dismutase, fibrogen, glucocerebrosidase, glutamate decarboxylase, human serum albumin, myelin basic protein, proinsulin, soluble CD4, lactoferrin, lactoglobulin, lysozyme, lactoalbumin, erythrpoietin, tissue plasminogen activator, human growth factor, antithrombin III, insulin, prolactin, and al-antitrypsin.

In a preferred embodiment, the nucleic acid is under the control of a promoter, e.g., a caprine or heterologous promoter. The promoter can be a tissue-specific promoter. The tissue specific promoter can be any of: a milk-specific promoter; a blood-specific promoter; a muscle-specific promoter; a neural-specific promoter; a skin-specific promoter; a hair specific promoter; and, a urine-specific promoter. The milk-specific promoter can be, e.g., any of: a β-casein promoter; a β-lactoglobin promoter; a whey acid protein promoter; and a lactalbumin promoter.

In a preferred embodiment, the somatic cell is a fibroblast. The fibroblast can be a primary fibroblast or a primary derived fibroblast.

S In a preferred embodiment, the cell is obtained from a goat, e.g., an embryonic or fetal goat, derived from a germ cell obtained from a transgenic goat. The germ cell can be sperm or an oocyte from a transgenic goat.

In a preferred embodiment, the cell is used as a source of genetic material for nuclear transfer.

In another aspect, the invention features, a method of preparing an embryonic or fetal caprine somatic cell line. The method includes inseminating a female recipient with the semen from a goat; obtaining a transgenic embryo from the recipient; obtaining a somatic cell from the embryo; and, culturing the cell in a suitable medium, such that a somatic cell line is obtained.

In a preferred embodiment, the cell line is a genetically engineered cell line, e.g., the cell comprises a transgene. The transgene can be: integrated into the somatic cell genome; a heterologous transgene, e.g., a heterologous transgene which includes a human sequence; a knockout, knockin or other event which disrupts the expression of a caprine gene; a sequence which encodes a protein, e.g., a human protein; a heterologous promoter; a heterologous sequence under the control of a promoter, e.g., a caprine promoter. The transgenic sequence can encode any product of interest such as a protein, polypeptide or peptide.

In a preferred embodiment, the transgene encodes any of: a hormone, an immunoglobulin, a plasma protein, and an enzyme. The transgene can encode any protein, e.g., any of: α-1 proteinase inhibitor, alkaline phosphotase, angiogenin, extracellular superoxide dismutase, fibrogen, glucocerebrosidase, glutamate decarboxylase, human serum albumin, myelin basic protein, proinsulin, soluble CD4, lactoferrin, lactoglobulin, lysozyme, lactoalbumin, erythrpoietin, tissue plasminogen activator, human growth factor, antithrombin III, insulin, prolactin, and α1-antitrypsin.

In a preferred embodiment, the transgene is under the control of a promoter, e.g., a caprine or heterologous promoter. The promoter can be a tissue-specific promoter. The tissue specific promoter can be any of: a milk-specific promoter; a blood-specific promoter; a muscle-specific promoter; a neural-specific promoter; a skin-specific promoter; a hair specific promoter; and, a urine-specific promoter. The milk-specific promoter can be, e.g., any of: a β-casein promoter; a β-lactoglobin promoter; a whey acid protein promoter; and a lactalbumin promoter.

In a preferred embodiment, the genetically engineered cell includes a nucleic acid, e.g., a nucleic acid encoding a polypeptide, which has been introduced into the cell. The nucleic acid can be: integrated into the genome of the somatic cell; a heterologous nucleic acid, e.g., a heterologous nucleic acid which includes a human sequence; a knockout, knockin or other event which disrupts the expression of a caprine gene; a sequence which encodes a protein, e.g., a human protein; a heterologous promoter; a heterologous sequence under the control of a promoter, e.g., a caprine promoter. The nucleic acid sequence can encode any product of interest such as a protein, polypeptide or peptide.

In a preferred embodiment, the nucleic acid can encodes any of: a hormone, an immunoglobulin, a plasma protein, and an enzyme. The nucleic acid can encode, e.g., any of: α-1 proteinase inhibitor, alkaline phosphotase, angiogenin, extracellular superoxide dismutase, fibrogen, glucocerebrosidase, glutamate decarboxylase, human serum albumin, myelin basic protein, proinsulin, soluble CD4, lactoferrin, lactoglobulin, lysozyme, lactoalbumin, erythrpoietin, tissue plasminogen activator, human growth factor, antithrombin III, insulin, prolactin, and α1-antitrypsin.

In a preferred embodiment, the nucleic acid is under the control of a promoter, e.g., a caprine or heterologous promoter. The promoter can be a tissue-specific promoter. The tissue specific promoter can be any of: a milk-specific promoter; a blood-specific promoter; a muscle-specific promoter; a neural-specific promoter; a skin-specific promoter; a hair specific promoter; and, a urine-specific promoter. The milk-specific promoter can be, e.g., any of: a β-casein promoter; a β-lactoglobin promoter; a whey acid protein promoter; and a lactalbumin promoter.

In a preferred embodiment, the somatic cell is a fibroblast. The fibroblast can be a primary fibroblast or a primary derived fibroblast.

In a preferred embodiment, the cell is used as a source of genetic material for nuclear transfer.

The terms protein, polypeptide and peptide are used interchangeably herein.

The term “genetically engineered”, as used herein, refers to a cell altered by human intervention, e.g., a transgenic cell or other cells into which a nucleic acid has been introduced.

As used herein, the term “transgenic sequence” refers to a nucleic acid sequence (e.g., encoding one or more human proteins), which is inserted by artifice into a cell. The transgenic sequence, also referred to herein as a transgene, can become part of the genome of an animal which develops in whole or in part from that cell. In embodiments of the invention, the transgenic sequence is integrated into the chromosomal genome. If the transgenic sequence is integrated into the genome it results, merely by virtue of its insertion, in a change in the nucleic acid sequence of the genome into which it is inserted. A transgenic sequence can be partly or entirely species-heterologous, i.e., the transgenic sequence, or a portion thereof, can be from a species which is different from the cell into which it is introduced. A transgenic sequence can be partly or entirely species- homologous, i.e., the transgenic sequence, or a portion thereof, can be from the same species as is the cell into which it is introduced. If a transgenic sequence is homologous (in the sequence sense or in the species-homologous sense) to an endogenous gene of the cell into which it is introduced, then the transgenic sequence, preferably, has one or more of the following characteristics: it is designed for insertion, or is inserted, into the cell's genome in such a way as to alter the sequence of the genome of the cell into which it is inserted (e.g., it is inserted at a location which differs from that of the endogenous gene or its insertion results in a change in the sequence of the endogenous endogenous gene); it includes a mutation, e.g., a mutation which results in misexpression of the transgenic sequence; by virtue of its insertion, it can result in misexpression of the gene into which it is inserted, e.g., the insertion can result in a knockout of the gene into which it is inserted. A transgenic sequence can include one or more transcriptional regulatory sequences and any other nucleic acid sequences, such as introns, that may be necessary for a desired level or pattern of expression of a selected nucleic acid, all operably linked to the selected nucleic acid. The transgenic sequence can include an enhancer sequence and or sequences which allow for secretion.

The term “heterologous promoter” as used herein, refers to a promoter which is not normally associated with the gene it controls or which is heterologous to the animal into which it is introduced.

The term “purified” as used herein, refers to a cell or preparation of cells which has been separated from its natural environment. This includes gross physical separation from a non-human animal, e.g., a goat, and alteration of a cell's relationship with neighboring cells with which it is direct contact with by, for example, dissociation. The cell can be alone or in a preparation of cells. A purified cell or cells should be substantially free of other cell types. As used herein, the term “preparation” refers to a group of two or more cells. For example, a purified preparation of embryonic cells or somatic cells refers to a preparation which includes at least 10, 20, 30, 40, 50, 60, 70, 80, 85, 90, 95% embryonic or somatic cells.

Other features of the invention will be apparent from the following description and from the claims.

### DETAILED DESCRIPTION OF THE INVENTION

Sources of Somatic Cells:

Somatic Cells

This invention features a purified somatic cell which can be used to prepare the cell line described herein. The term “somatic cell”, as used herein, refers to a differentiated cell. The cell can be a somatic cell or a cell that is committed to a somatic cell lineage.

The somatic cell can be taken from an animal at any stage of development, e.g., an embryo, a fetus or an adult. Embryonic cells are preferred. Preferably, the embryonic cells are committed to somatic cell lineage. Embryonic cells committed to a somatic cell lineage refer to cells isolated on or after day 10 of embryogenesis.

The cell line can include primary cells, primary-derived cells, or both. The term “primary-derived cells” refer to primary cells which have undergone at least one subsequent division.

Suitable somatic cells include fibroblasts (e.g., primary fibroblasts, e.g., embryonic primary fibroblasts), muscle cells (e.g., myocytes), cumulus cells, neural cells, and mammary cells. Other suitable cells include hepatocytes and pancreatic islets. Preferably, the somatic cell is an embryonic somatic cell, e.g., a cell isolated on or after day 10 of embryogenesis. The genome of the somatic cells can be the naturally occurring genome or the genome can be genetically altered to comprise a transgenic sequence.

Somatic cells can be obtained by, for example, dissociation of tissue, e.g., by mechanical (e.g., chopping, mincing) or enzymatic means (e.g., trypsinization) to obtain a cell suspension and then by culturing the cells until a confluent monolayer is obtained. The somatic cells can then be harvested and prepared for cryopreservation, or maintained as a stock culture. The isolation of caprine somatic cells, e.g., fibroblasts, is described herein.

The somatic cell can be a quiescent or non-quiescent somatic cell. “Non-quiescent”, as used herein, refers to a cell in mitotic cell cycle. The mitotic cell cycle has four distinct phases, G₁, S, G₂ and M. The beginning event in the cell cycle, called START, takes place during the G₁ phase. “START” as used herein refers to early G₁ stage of the cell cycle prior to the commitment of a cell to proceeding through the cell cycle. The decision as to whether the cell will undergo another cell cycle is made at START. Once the cell has passed through START, it passes through the remainder of the G₁ phase (i.e., the pre-DNA synthesis stage). The S phase is the DNA synthesis stage, which is followed by the G₂ phase, the stage between synthesis and mitosis. Mitosis takes place during the M phase. If at START, the cell does not undergo another cell cycle, the cell becomes quiescent. In addition, a cell can be induced to exit the cell cycle and become quiescent. A “quiescent” cell, also referred to as a cell in G₀ phase, refers to a cell which is not in any of the four phases of the cell cycle. Preferably, the somatic cell is a cell in the G₀phase or the G₁ phase of the mitotic cell cycle.

Methods of determining which phase of the cell cycle a cell is in are known. For example, various markers are present at different stages of the cell cycle. Such markers can include cyclins D 1, 2, 3 and proliferating cell nuclear antigen (PCNA) for G₁, and BrDu to detect DNA synthetic activity. In addition, cells can be induced to enter the G₀ stage by culturing the cells on serum-deprived medium. Alternatively, cells in G₀ stage can be induced to enter the cell cycle, i.e., at G₁ stage, by serum activation.

Sources of Genetically Engineered Somatic Cells:

Transgenic Mammals

Methods for generating non-human transgenic mammals which can be used as a source of somatic cells in the invention are known in the art. Such methods can involve introducing DNA constructs into the germ line of a mammal to make a transgenic mammal. For example, one or several copies of the construct may be incorporated into the genome of a mammalian embryo by standard transgenic techniques.

Although goats are a preferred source of genetically engineered somatic cells, other non-human mammals can be used. Preferred non-human mammals are ruminants, e.g., cows, sheep, camels or goats. Goats of Swiss origin, e.g., the Alpine, Saanen and Toggenburg breed goats, are useful in the methods described herein. Additional examples of preferred non-human animals include oxen, horses, llamas, and pigs.

Preferably, the somatic cells are obtained from a transgenic goat. Methods of producing transgenic goats are known in the art. For example, a transgene can be introduced into the germline of a goat by microinjection as described, for example, in Ebert et al. (1994) Bio/Technology 12:699, hereby incorporated by reference.

Other transgenic non-human animals to be used as a source of genetically engineered somatic cells can be produced by introducing a transgene into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor.

DNA Constructs

A cassette which encodes a heterologous protein can be assembled as a construct which includes a promoter for a specific tissue, e.g., for mammary epithelial cells, e.g., a casein promoter, e.g., a goat beta casein promoter, a milk-specific signal sequence, e.g., a casein signal sequence, e.g., a β-casein signal sequence, and a DNA encoding the heterologous protein.

The construct can also include a 3′ untranslated region downstream of the DNA sequence coding for the non-secreted protein. Such regions can stabilize the RNA transcript of the expression system and thus increases the yield of desired protein from the expression system. Among the 3′ untranslated regions useful in the constructs for use in the invention are sequences that provide a poly A signal. Such sequences may be derived, e.g., from the SV40 small t antigen, the casein 3′ untranslated region or other 3′ untranslated sequences well known in the art. In one aspect, the 3′ untranslated region is derived from a milk specific protein. The length of the 3′ untranslated region is not critical but the stabilizing effect of its poly A transcript appears important in stabilizing the RNA of the expression sequence.

Optionally, the construct can include a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

The construct can also include about 10%, 20%, 30%, or more of the N-terminal coding region of a gene preferentially expressed in mammary epithelial cells. For example, the N-terminal coding region can correspond to the promoter used, e.g., a goat β-casein N-terminal coding region.

The construct can be prepared using methods known in the art. The construct can be prepared as part of a larger plasmid. Such preparation allows the cloning and selection of the correct constructions in an efficient manner. The construct can be located between convenient restriction sites on the plasmid so that they can be easily isolated from the remaining plasmid sequences for incorporation into the desired mammal.

Tissue-Specific Expression of Proteins

It is often desirable to express a protein, e.g., a heterologous protein, in a specific tissue or fluid, e.g., the milk, of a transgenic animal. The heterologous protein can be recovered from the tissue or fluid in which it is expressed. For example, it is often desirable to express the heterologous protein in milk. Methods for producing a heterologous protein under the control of a milk specific promoter are described below. In addition, other tissue-specific promoters, as well as, other regulatory elements, e.g., signal sequences and sequence which enhance secretion of non-secreted proteins, are described below.

Milk Specific Promoters

Useful transcriptional promoters are those promoters that are preferentially activated in mammary epithelial cells, including promoters that control the genes encoding milk proteins such as caseins, beta lactoglobulin (Clark et al., (1989) Bio/Technology-7: 487-492), whey acid protein (Gordon et al. (1987) Bio/Technology 5: 1183-1187), and lactalbumin (Soulier et al., (1992) FEBS Letts. 297: 13). Casein promoters may be derived from the alpha, beta, gamma or kappa casein genes of any mammalian species; a preferred promoter is derived from the goat beta casein gene (DiTullio, (1992) Bio/Technology 10:74-77). Milk-specific protein promoter or the promoters that are specifically activated in mammary tissue can be derived from cDNA or genomic sequences. Preferably, they are genomic in origin.

DNA sequence information is available for the mammary gland specific genes listed above, in at least one, and often in several organisms. See, e.g., Richards et al., J. Biol. Chem. 256, 526-532 (1981) (α-lactalbumin rat); Campbell et al., Nucleic Acids Res. 12, 8685-8697 (1984) (rat WAP); Jones et al., J. Biol. Chem. 260, 7042-7050 (1985) (rat β-casein); Yu-Lee & Rosen, J. Biol. Chem. 258, 10794-10804 (1983) (rat γ-casein); Hall, Biochem. J. 242, 735-742 (1987) (α-lactalbumin human); Stewart, Nucleic Acids Res. 12, 389 (1984) (bovine αs1 and κ casein cDNAs); Gorodetsky et al., Gene 66, 87-96 (1988) (bovine β casein); Alexander et al., Eur. J. Biochem. 178, 395-401 (1988) (bovine κ casein); Brignon et al., FEBS Lett. 188, 48-55 (1977) (bovine αS2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine β lactoglobulin); Vilotte et al., Biochimie 69, 609-620 (1987) (bovine α-lactalbumin). The structure and function of the various milk protein genes are reviewed by Mercier & Vilotte, J. Dairy Sci. 76, 3079-3098 (1993) (incorporated by reference in its entirety for all purposes). If additional flanking sequence are useful in optimizing expression of the heterologous protein, such sequences can be cloned using the existing sequences as probes. Mammary-gland specific regulatory sequences from different organisms can be obtained by screening libraries from such organisms using known cognate nucleotide sequences, or antibodies to cognate proteins as probes.

Signal Sequences

Useful signal sequences are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the milk-specific promoter used in the construct, which are described below. The size of the signal sequence is not critical. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin can be used. A preferred signal sequence is the goat β-casein signal sequence.

Signal sequences from other secreted proteins, e.g., proteins secreted by kidney cells, pancreatic cells or liver cells, can also be used. Preferably, the signal sequence results in the secretion of proteins into, for example, urine or blood.

Amino-Terminal Regions of Secreted Proteins

A non-secreted protein can also be modified in such a manner that it is secreted such as by inclusion in the protein to be secreted of all or part of the coding sequence of a protein which is normally secreted. Preferably the entire sequence of the protein which is normally secreted is not included in the sequence of the protein but rather only a sufficient portion of the amino terminal end of the protein which is normally secreted to result in secretion of the protein. For example, a protein which is not normally secreted is fused (usually at its amino terminal end) to an amino terminal portion of a protein which is normally secreted.

In one aspect, the protein which is normally secreted is a protein which is normally secreted in milk. Such proteins include proteins secreted by mammary epithelial cells, milk proteins such as caseins, beta lactoglobulin, whey acid protein, and lactalbumin. Casein proteins include alpha, beta, gamma or kappa casein genes of any mammalian species. A preferred protein is beta casein, e.g., goat beta casein. The sequences which encode the secreted protein can be derived from either cDNA or genomic sequences. Preferably, they are genomic in origin, and include one or more introns.

Other Tissue-Specific Promoters

Other tissue-specific promoters which provide expression in a particular tissue can be used. Tissue specific promoters are promoters which are expressed more strongly in a particular tissue than in others. Tissue specific promoters are often expressed essentially exclusively in the specific tissue.

Tissue-specific promoters which can be used include: a neural-specific promoter, e.g., nestin, Wnt-1, Pax-1, Engrailed-1, Engrailed-2, Sonic hedgehog; a liver-specific promoter, e.g., albumin, alpha-1 antirypsin; a muscle-specific promoter, e.g., myogenin, actin, MyoD, myosin; an oocyte specific promoter, e.g., ZP1, ZP2, ZP3; a testes-specific promoter, e.g., protamin, fertilin, synaptonemal complex protein-1; a blood-specific promoter, e.g., globulin, GATA-1, porphobilinogen deaminase; a lung-specific promoter, e.g., surfactant protein C; a skin- or wool-specific promoter, e.g., keratin, elastin; endothelium-specific promoters, e.g., Tie-1, Tie-2; and a bone-specific promoter, e.g., BMP.

In addition, general promoters can be used for expression in several tissues. Examples of general promoters include β-actin, ROSA-21, PGK, FOS, c-myc, Jun-A, and Jun-B.

Heterologous Proteins

Transgenic sequences encoding heterologous proteins can be introduced into the germline of a non-human mammal as described above.

The protein can be a complex or multimeric protein, e.g., a homo- or heteromultimer, e.g., proteins which naturally occur as homo- or heteromultimers, e.g., homo- or hetero-dimers, trimers or tetramers. The protein can be a protein which is processed by removal, e.g., cleavage, of N-terminus, C-terminus or internal fragments. Even complex proteins can be expressed in active form. Protein encoding sequences which can be introduced into the genome of mammal, e.g., goats, include glycoproteins, neuropeptides, immunoglobulins, enzymes, peptides and hormones. The protein may be a naturally occurring protein or a recombinant protein, e.g., a fragment, fusion protein, e.g., an immunoglogulin fusion protein, or mutien. It may be human or non-human in origin. The heterologous protein may be a potential therapeutic or pharmaceutical agent such as, but not limited to: alpha-1 proteinase inhibitor, alpha-1 antitrypsine, alkaline phosphatase, angiogenin, antithrombin III, any of the blood clotting factors including Factor VIII, Factor IX, and Factor X chitinase, erythropoietin, extracellular superoxide dismutase, fibrinogen, glucocerebrosidase, glutamate decarboxylase, human growth factor, human serum albumin, immunoglobulin, insulin, myelin basic protein, proinsulin, prolactin, soluble CD4 or a component or complex thereof, lactofernin, lactoglobulin, lysozyme, lactalbumin, tissue plasminogen activator or a variant thereof.

Immunoglobulins are particularly preferred heterologous protiens. Examples of immunoglobulins include IgA, IgG, IgE, IgM, chimeric antibodies, humanized antibodies, recombinant antibodies, single chain antibodies and antibody-protein fusions.

Nucleotide sequence information is available for several of the genes encoding the heterologous proteins listed above, in at least one, and often in several organisms. See e.g., Long et al. (1984) Biochem. 23(21):4828-4837 (aplha-1 antitrypsin); Mitchell et al. (1986) Prot. Natl. Acad. Sci USA 83:7182-7186 (alkaline phosphatase); Schneider et al. (1988) EMBO J. 7(13):4151-4156 (angiogenin); Bock et al. (1988) Biochem. 27(16):6171-6178 (antithrombin III); Olds et al. (1991) Br. J. Haematol. 78(3):408-413 (antithrombin III); Lin et al. (1985) Proc. Natl. Acad. Sci. USA 82(22):7580-7584 (erythropoeitin); U.S. Pat. No. 5,614,184 (erythropoietin); Horowitz et al. (1989) Genomics 4(1):87-96 (glucocerebrosidase); Kelly et al. (1992) Ann. Hum. Genet. 56(3):255-265 (glutamte decarboxylase); U.S. Pat. No. 5,707,828 (human serum albumin); U.S. Pat. No. 5,652,352 (human serum albumin); Lawn et al. (1981) Nucleic Acid Res. 9(22):6103-6114 (human serum albumin); Kamholz et al. (1986) Prot. Natl. Acad. Sci. USA 83(13):4962-4966 (myelin basic protein); Hiraoka et al. (1991) Mol. Cell Endocrinol. 75(1):71-80 (prolactin); U.S. Pat. No. 5,571,896 (lactoferrin); Pennica et al. (1983) Nature 301(5897):214-221 (tissue plasminogen activator); Sarafanov et al. (1995) Mol. Biol. 29:161-165, the contents of which are incorporated herein by reference.

This invention is further illustrated by the following examples which in no way should be construed as being further limiting. The contents of all cited references (including literature references, issued patents, published patent applications, and co- pending patent applications) cited throughout this application are incorporated by reference.

### EXAMPLES

Donors and recipients used in the following examples were dairy goats of the following breeds (mixed or not): Alpine, Saanen, and Toggenburg. All goats were maintained at the Genzyme Transgenics farm in Charlton, Mass. Collections and transfers were completed during the spring and early summer (off-season).

Isolation of Caprine Somatic Cells

Caprine fetal fibroblast cell lines used as karyoplast donors were derived from six day 35-40 fetuses produced by artificially inseminating non-transgenic does with fresh collected semen from a transgenic antithrombin III (ATIII) founder buck. An ATIII cell line was chosen since it provides a well characterized genetic marker to the somatic cell lines, and it targets high level expression of a complex glycosylated protein (ATIII) in the milk of lactating does. Three fetuses which were derived from the semen of the transgenic ATIII buck were surgically removed at day 40 post coitus and placed in equilibrated Ca⁺⁺/Mg⁺⁺-free phosphate buffered saline (PBS). Cell suspensions were prepared by mincing and digesting fetal tissue in 0.025% trypsin/0.5 mM EDTA at 37° C. for ten minutes. Cells were washed with equilbrated Medium 199™ (M199)(Gibco)+10% Fetal Bovine Serum (FBS) supplemented with nucleosides, 0.1 mM 2-mercaptoethanol, 2 mM L-glutamine, 1% penicillin/streptomycin (10,000 I.U. each/ml) (fetal cell medium), and cultured in 25 cm² flasks. The cultures were re-fed 24 hours later with equilibrated fetal cell medium. A confluent monolayer of primary fetal cells was harvested by trypsinization on day four by washing the monolayer twice with Ca⁺⁺/Mg⁺⁺-free PBS, followed by incubation with 0.025% trypsin/0.5 mM EDTA at 38° C. for 7 minutes.

Cells potentially expressing ATIII were then prepared for cryopreservation, or maintained as stock cultures.

Sexing and Genotyping of Donor Cell Lines

Genomic DNA was isolated from fetal head tissue for ATIII donor karyoplasts by digestion with proteinase K followed by precipitation with isopropanol as described in Laird et al. (1991) Nucleic Acid Res. 19:4293, and analyzed by polymerase chain reaction (PCR) for the presence of human Antithrombin III (ATIII) sequences as well as for sexing. The ATIII sequence is part of the BC6 construct (Goat Beta-Casein—human ATIII cDNA) used to generate the ATIII transgenic line as described in Edmunds et al. (1998) Blood 91:4561-4571. The human ATIII sequencewas detected by amplification of a 367 bp sequence with oligonucleotides GTC11 and GTC12 (see below). For sexing, the zfX/zfY primer pair was used (see below) giving rise to a 445 bp (zfX)/447 bp (zfy) doublet. Upon digestion with the restriction enzyme SacI (New England Biolabs), the zfX band was cut into two small fragments (272 and 173 bp). Males were identified by the presence of the uncut 447 bp zfY band.

For the PCR reactions, approximately 250 ng of genomic DNA was diluted in 50 ml of PCR buffer (20 mM Tns pH 8.3, 50 mM KCl and 1.5 mM MgCl₂, 0.25 mM deoxynucleotide triphosphates, and each primer at a concentration of 600 mM) with 2.5 units of Taq polymerase and processed using the following temperature program:

| | | |
|---|---|---|
| 1 cycle at | 94° C. | 60 seconds |
| 5 cycles at | 94° C. | 30 seconds |
| | 58° C. | 45 seconds |
| | 74° C. | 45 seconds |
| 30 cycles at | 94° C. | 30 seconds |
| | 55° C. | 30 seconds |
| | 74° C. | 30 seconds |

The following primer set was used to detect the human ATIII sequence:

| | |
|---|---|
| GTC 11: CTCCATCAGTTGCTGGAGGGTGTCATTA | (SEQ ID NO:1) |
| GTC 12: GAAGGTTTATCTTTTGTCCTTGCTGCTCA | (SEQ ID NO:2) |

The following primer set was used for sexing:

| | | |
|---|---|---|
| zfX: | ATAATCACATGOAGAGCCACAAGC | (SEQ ID NO:3) |
| zfY: | GCACTTCTTTGGTATCTGAGAAAG | (SEQ ID NO:4) |

Two of the fetuses were identified to be male and were both negative for the ATIII sequence. Another fetus was identified as female and confirmed positive for the presence of the ATIII sequence.

Preparation of ATIII-Expressing Donor Cells for Embryo Reconstitution

A transgenic female line (CFF155-92-6) originating from a day 40 fetus was identified by PCR analyses, as described above, and used for all nuclear transfer manipulations. Transgenic fetal fibroblast cells were maintained in 25 cm² flasks with fetal cell medium, re-fed on day four following each passage, and harvested by trypsinization on day seven. From each passage, a new 25 cm² flasks was seeded to maintain the stock culture. Briefly, fetal cells were seeded in 4-well plates with fetal cell medium and maintained in culture (5% CO₂ at 39° C.). Forty-eight hours later, the medium was replaced with fresh fetal cell medium containing 0.5% FBS. The culture was re-fed every 48-72 hours over the next seven days with fresh fetal cell medium containing 0.5% FBS. On the seventh day following first addition of fetal cell medium (0.5% FBS), somatic cells used as karyoplast donors were harvested by trypsinization as previously described. The cells were resuspended in equilibrated M199+10% FBS supplemented with 2 mM L-glutamine, 1% penicillin/streptomycin (10,000 I.U. each/ml).

All patents and other references cited herein are incorporated by reference. Other embodiments are within the following claims:

1-91. (canceled)

92. A method for the accelerated production of transgenic animals comprising:
a) transfecting a first non-human differentiated somatic cell or cell-line with a transgene construct containing a first DNA sequence;
b) selecting a transfected cell or cell-line into which said first DNA sequence has been inserted into the genome of said first non-human differentiated somatic cell or cell-line;
c) performing a first nuclear transfer procedure to generate a first transgenic animal at least heterozygous for said first DNA sequence;
d) performing a biopsy or other cell selection technique to obtain cells to establish a second non-human differentiated somatic cell or cell-line from said first transgenic animal;
e) characterizing said second non-human differentiated somatic cell or cell-line using known molecular biology methods to ensure that the selected said second non-human differentiated somatic cell or cell-line is at least heterozygous for said first DNA sequence; and
f) performing a second nuclear transfer procedure with at least one of said second non-human differentiated somatic cells to produce at least a second transgenic animal at least heterozygous for said first DNA sequence.

93. The method of claim 92, wherein said first transgenic animal is at an embryonic stage of development.

94. The method of claim 92, wherein said first transgenic animal is at a fetal stage of development.

95. The method of claim 92, further comprising developing said first transgenic animal into an adult non-human animal.

96. The method of claim 92, wherein said first transgenic animal is a mammal.

97. The method of claim 92, wherein said first DNA sequence encodes a desired protein;

98. The method of claim 92, wherein the genetic composition of said first transgenic animal is characterized to confirm the presence and expression of the transgene.

99. The method of claim 92, wherein said first nuclear transfer procedure further comprises transferring the nucleus of said transfected cell into a suitable enucleated recipient cell of the same species, thereby obtaining a reconstituted cell.

100. The method of claim 92, wherein said first transgenic animal is biopsied so as to characterize the genome of said first transgenic animal.

101. The method of claim 92, wherein at least one of the cells from said second non- human differentiated somatic cell or cell-line is expanded through cell culture techniques for use in said second round of nuclear transfer so as to produce a multiplicity of animals transgenic for said DNA of interest.

102. The method of claim 96, wherein the source of said differentiated somatic cell or cell-line is an ungulate.

103. The method of either claims 102, wherein said differentiated somatic cell or cell- line is from an ungulate selected from the group consisting of bovine, ovine, porcine, equine, caprine and buffalo.

104. A method of preparing a genetically engineered transgenic mammal, comprising:
(a) inseminating a first female non-human mammal recipient with semen from a transgenic non-human animal of the same species known to have a transgene present and expressed;
(b) obtaining a transgenic non-human embryo from said first female recipient;
(c) obtaining a somatic cell from said embryo;
(d) culturing said differentiated somatic cell in a suitable medium, such that a differentiated somatic cell line is obtained and,
(e) performing a nuclear transfer procedure with said non-human differentiated somatic cells to produce at least one transgenic mammal at least heterozygous for said first DNA sequence;
wherein said first DNA sequence encoding a desired gene is actuated by a tissue specific promoter.

105. The resultant offspring of the methods of claim 104.

106. The method of claim 92, wherein said second non-human differentiated somatic cell or cell-line cells are obtained from an embryonic goat on or after day 10 of embryogenesis.

107. The method of claim 92, wherein said second non-human differentiated somatic cell or cell line preparation is kept in an airtight container.

108. The method of claim 92 wherein said first DNA sequence codes for a biopharmaceutical protein product.

109. The method of claim 108 wherein said first DNA sequence encoding a desired gene is actuated by at least one beta casein promoter.

110. The resultant milk derived from the offspring of the methods of claim 108.

111. The method of claim 92, wherein said second non-human differentiated somatic cell or cell-line is obtained from said first transgenic animal by known tissue dissociation means including enzymatic means and/or mechanical means.

112. The method of claim 92, wherein said second non-human differentiated somatic cell or cell-line is selected from a group of cell types present in said first transgenic animal including:
a) fibroblasts
b) cumulus cells
c) neural cells
d) mammary cells; and
e) myocytes.

113. The resultant offspring of the methods of claim 92.

114. The method of claim 104 wherein said transgene codes for a biopharmaceutical protein product.

115. The method of claim 114 wherein said tissue specific promoter is a beta casein promoter.

116. The resultant milk derived from the offspring of the methods of claim 114.

117. The method of claim 104, wherein said second non-human differentiated somatic cell or cell-line is obtained from said first transgenic animal by known tissue dissociation means including enzymatic means and/or mechanical means.

118. The method of claim 104, wherein said second non-human differentiated somatic cell or cell-line is selected from a group of cell types present in said first transgenic animal including:
a) fibroblasts
b) cumulus cells
c) neural cells
d) mammary cells; and
e) myocytes.

119. The method of claim 92, wherein said transgene construct comprises a nucleic acid sequence encoding a human polypeptide.

120. The method of claim 92, wherein said transgene construct is capable of knocking out the expression of a gene endogenous to said first transgenic animal.

121. The method of claim 119, wherein said transgene construct further comprises a promoter wherein the nucleic acid is under the control of said promoter.

122. The method of claim 121, wherein said promoter is a tissue specific promoter.

123. The method of claim 122, wherein said tissue-specific promoter is a promoter preferentially expressed in mammary gland epithelial cells.

124. The method of claim 123, wherein said promoter is selected from the group consisting of a β-casein promoter, β-lactoglobin promoter, whey acid protein promoter and lactalbumin promoter.

125. The method of claim 121, wherein said promoter is a caprine promoter.

126. The method of claim 119, wherein said nucleic acid encodes a polypeptide selected from the group consisting of a hormone, an immunoglobin, a plasma protein, and an enzyme.

127. The method of claim 119, wherein said nucleic acid encodes a polypeptide selected from the group consisting of an α-1 proteinase inhibitor, an alkaline phosphotase, an angiogenin, an extracellular superoxide dismutase, a fibrogen, a glucocerebrosidase, a glutamate decarboxylase, a human serum albumin, a myelin basis protein, a proinsulin, a soluble CD4, a lactoferrin, a lactoglobulin, a lysozyme, a lactoalbumin, an erythropoietin, a tissue plasminogen activator, a human growth factor, an antithrombin III, an insulin, a prolactin, and an α-1-antitrypsin.

128. The method of claim 92, wherein said second non-human differentiated somatic cell or cell-lines are fibroblasts.

129. The method of claim 128, wherein said fibroblasts are primary fibroblasts.

130. The method of claim 128, wherein said fibroblasts are primary derived fibroblasts.
